(19) United States
(12) Patent Application Publication
(10) Pub. No.: US 2026/0341462 A1
(43) Pub. Date: Oct. 29, 2026

(54) METHOD AND SYSTEM FOR THE PERSONALIZED MANUFACTURING OF NUTRITIONAL SUPPLEMENTS

(71) Applicant: INDYVIT AG, Reinach (CH)

(72) Inventor: Philipp KIRSCHBAUM, Biel-Benken (DE)

(21) Appl. No.: 19/107,396

(22) PCT Filed: Jan. 3, 2023
(86) PCT No.: PCT/IB2023/050028

§ 371 Date: Feb. 27, 2025

(30) Foreign Application Priority Data
Aug. 31, 2022 (CH) CH001016/2022

(51) Int. Cl. G16H 20/60; A23L 33/135; A23L 33/15; G16H 10/40

(52) U.S. Cl. CPC G16H 20/60; A23L 33/135; A23L 33/15; G16H 10/40

(57) ABSTRACT

The invention relates to a method, wherein a nutritional supplement is a preparation of N ingredients, the method comprises: providing a carrier having a first defined volume V; providing a first ingredient I1 having a first density dI1 and a first concentration kI1; defining an ingredient amount mI1 of the first ingredient I1 on the basis of parameters, wherein the parameters are customized for the personalized manufacturing process of a preparation; repeating the two last steps for the second ingredient I2 and any additional ingredient In, wherein the manufacturing process comprises filling a total amount of ingredients, and, for each ingredient, a filling amount a1n is calculated from the corresponding ingredient amount mIn divided by the corresponding density dIn and the corresponding concentration kIn (mIn/dIn/kIn), and the total of all N filling amounts a1n is divided by the defined volume V of the carrier (ΣaIn/V).

The present invention relates to a method and a system for the personalized manufacturing of nutritional supplements and/or nutritional supplement mixtures, each according to the preambles of the independent claims. The invention further relates to a computer program for the carrying out of the method according to the invention.

### TECHNOLOGICAL BACKGROUND

Nutritional supplements, often abbreviated as NS, are food products that are intended to supplement the human metabolism with certain nutrients, such as vitamins, vitaminoids or minerals. These nutrients are contained in concentrated and dosed form, which is why they are often presented in a form that is not typical for food, such as tablets, bottled drinks, powders, effervescent tablets or capsules. Also known and frequently used are antioxidants, essential and/or non-essential amino acids, protein mixtures and/or concentrates or isolates, carnitine, creatine, and the like.

Nutritional supplements are individually packaged, i.e. a specific nutritional supplement is commercially available as a packaged product at a specific dosage.

While being manufactured, a new unit is created by combining different substances or ingredients.

Incorrect or unbalanced nutrition can lead to deficiencies, as can a lack of sun or a purely vegan diet. In these cases taking nutritional supplements is a suitable supplementation or compensation in order to reach the recommended daily amount. Taking nutritional supplements can also be beneficial in certain living situations, or for particular risk groups.

There are combination preparations that can contain a number of nutritional supplements, sometimes with different doses, and that are sometimes marketed to a specific group, such as athletes, women, people over 60, and the like.

The thing that previous forms of administration and distribution of these nutritional supplements have in common is that they are not tailored to the individual needs of a specific user.

If one wants to take an individual nutritional supplement regimen, they have to resort to individually packaged produced nutritional supplements and create an individual regimen for taking them. This means that a person must take a large number of tablets, drinks or capsules. In addition to the obvious impractical aspects of such a large number of capsules, this complexity also means that the nutritional supplement regimen will be poorly adhered to, so that the hoped for benefits do not result or only do so to a very limited extent.

In addition, this type of dosing can pose a heath risk, since the end consumer often takes a disproportionate amount of fillers and excipients with each nutritional supplement preparation. As for preparations with overlapping nutritional supplements, it is often difficult for the end consumers to estimate what dose they are actually taking.

Therefore, there is a need for a method and system for the personalized manufacturing of nutritional supplements of the kind indicated above, which comprises a high degree of customization options, and which should ensure that the maximum values prescribed by health authorities or that are personally defined, are adhered to.

### PRESENTATION OF THE INVENTION

One purpose of the invention is to overcome at least one of known drawbacks. A particular purpose of the invention is to provide a method and system of the type mentioned above, which enables the safe and efficient manufacturing of an individual nutritional supplement for an end consumer.

This purpose is achieved by a method and/or system according to the characterizing part of the independent claims.

One aspect of the present disclosure relates to a method for personalized manufacturing of nutritional supplements, in particular for standardized personalized manufacturing of nutritional supplements.

A method for the personalized manufacturing of a nutritional supplement, wherein the nutritional supplement is a preparation of N ingredients and N is an integer multiple of 1, may comprise the following steps, such as preparing at least one carrier with a first defined volume V; preparing a first ingredient I₁ with a first density dI₁ and a first concentration kI₁; defining an ingredient amount mI₁ of the first ingredient I₁ by means of a number of parameters, wherein the parameters are individual for the personalized manufacturing of a preparation. The two steps indicated above are repeated for a second ingredient I₂ and for each additional ingredient Iₙ. The manufacturing comprises a filling of a total amount of N ingredients, where for each ingredient Iₙ a filling amount aIₙ is determined from the respective ingredient amount mIₙ, divided by the respective density dIₙ and the respective concentration kIₙ (mIₙ/dIₙ/kIₙ) and total of all N filling amounts aIₙ is divided by the defined volume V of the carrier (ΣaIₙ/V).

The method is flexible as to the number N of ingredients as well as to the selection of the respective ingredients.

In a particular embodiment, the ingredient amount mIₙ is defined on the basis of parameters that are determined depending on consumer input. The definition or determination of the amount of ingredients can advantageously be done as, or in response to, consumer input. This has the advantage that targeted input can be requested from the consumer. This input can reflect behaviors, preferences or conditions that lead to the individualized manufacturing.

Alternatively and/or additionally, the ingredient amount mIn is defined on the basis of parameters that are determined depending on a measurement made on or in the body. The definition of the ingredient amount can accordingly be made in response to this measurement. This has the advantage that objective deficiencies can be remedied with an appropriate ingredient composition. It is advantageous if the parameters are indicative of one or more attributes of a consumer for whom the preparation is created, since the preparation can the be specifically matched.

Each parameter can be selected as indicative for one or more attributes from a group or subgroup. Such groups may include: age, sex, body mass index, dietary habits, sleep rhythm, working hours, media consumption, leisure behavior, weight, height, skin color, eye color, allergies and/or intolerances, resting heart rate, blood oxygen saturation, cortisol levels, drug and/or stimulant use, blood count information, blood sugar, movement and/or activity data, such as calorie consumption from wearables.

In the case of the ingredients Iₙ, wherein N is a whole number between 2 and 20, preferably between 7 and 15, and more preferably is 11. 9 can also be suitable. If the number is too small, the desired effect may not occur. If the number of ingredients is too large, complexity also increases.

If a flavor additive is taken into account for the overall filling amount of ingredients Iₙ, the nutritional supplement becomes more pleasant for the consumer. The added flavor may comprise aromas such as peppermint, citrus and vanilla.

If one or more ingredients are selected in such a way that they correspond to a percentage of a recommended daily value, then the ingredients are well tolerated when taken and are within the recommended values.

In one particular embodiment, the ingredient Iₙ and/or vital substance is a substance selected from the group consisting of: cholecalciferol, cyanocobalamin, calcium D-pantothenate, menaquinone-7, black pepper powder, ginseng root powder, turmeric root extract, guarana, L-ascorbic acid, magnesium oxide, maltodextrin. The selected ingredients can in general come from amino acids, minerals, plant substances, vitamins or other substances.

In a further embodiment, a probiotic is provided as ingredient Iₙ. Living microorganisms can be included in the preparation for this purpose. These are considered functional food products. If consumed in sufficient quantities, probiotics can have a health-promoting effect on the host organism.

Suitable microorganisms can include probiotic bacterial strains of bifidobacteria and/or lactobacilli, which are approved as nutritional supplements. Certain yeast strains are also used as probiotics. Corresponding probiotics can be provided as a powder for use as an ingredient according to the present invention, with lyophilizates commonly being used.

In one particular embodiment, the ingredient is a prebiotic. Prebiotics can promote the growth and/or existence of a specific microorganism or group of microorganisms in the intestines by supplying them with usable food. Likewise, prebiotics include substances that change the environment in such a way that environmental parameters are conducive to the growth and maintenance of a specific population of microorganisms. In one particularly preferred embodiment, probiotics and prebiotics are provided as ingredients in a synergistic relationship with each other. For example, certain bacterial strains can be selected that are able to break down and use certain types of sugar, or that can have a competitive advantage compared to other microorganisms when certain types of sugar are added. In a particularly preferred embodiment, the prebiotics are oligosaccharides. In a preferred embodiment, the carrier is a capsule, in particular a two-part capsule made essentially of gelatin or hydroxypropyl methylcellulose. The capsule can easily be filled with the specified total amount. It is likewise easy for the consumer to take the capsule, since it is easy to swallow and the gelatin dissolves easily in the body. The capsule is preferably designed so that it only dissolves where the effect is best. In addition, the consumer has the option of opening the capsule and taking the ingredients directly in powder form.

Alternatively and/or additionally, the carrier can also be prepared as a tablet, pill, gummy, bottled drink, effervescent tablet and/or in another form. The individualized nutritional supplement can also be dispensed in powder form, packaged in small bags or pouches, such as a sealed bag or sachet.

For certain consumers or groups of consumers, an oil or liquid may be provided, which is intended to be taken with the carrier. This can result in intended advantageous swallowing or dissolving effects.

It can also be advantageous to provide multiple carriers with different ingredients. This allows a larger nutritional supplement range to be covered.

In one particular embodiment, the ingredient Iₙ is a substance selected from the group consisting of: biotin, folate, niacin, pantothenic acid, riboflavin (vitamin B2), thiamine (vitamin B1), vitamin B6, vitamin B12, vitamin C, vitamin D3, vitamin E, calcium, magnesium, turmeric, ginger, fenugreek, organic sencha green tea, beta carotene (provitamin A), vitamin K2, iron, selenium, zinc, copper, iodine, chromium, molybdenum, manganese, potassium, fluoride, chloride, phosphorus, lactase, L-arginine, L-glutamine, L-histidine, L-lysine, coenzyme Q10, caffeine, glucosamine, creatine, L-carnitine, taurine, garlic, black pepper, organic maca powder, spirulina, cannabidiol, ginseng, oligomeric proanthocyanidins, guarana, echinacea, green coffee, barley grass, saffron and L-tryptophan.

The ingredient can be in a suitable form of administration, such as an extract, salt, hydrolysate, lyophilizate, and the like.

The ingredient can be provided in a form intended as a nutritional supplement. This may be regulated by the law (e.g. in that they do not exert any pharmacological effect). For the purposes of the present invention, nutritional supplements can be defined as foods intended to supplement a normal diet. These may comprise single or multiple concentrates of vitamins, minerals or other substances with a nutritionally specific or physiological effect, and put on the market in a dosed form.

Alternatively and/or additionally, the ingredient Iₙ can be administered in a form that is active on various molecular target structures in the organism, for example as a phytopharmaceutical. Phytopharmaceuticals suitable according to the invention are, for example, dried, cut or whole plant parts, which can originate from one or more plants, for example selected from the group consisting of valerian, bearberry, birch, nettle, goldenrod, St. John's wort, chamomile, lavender, mallow, passionflower, peppermint, sage, senna, licorice, thyme, hawthorn and/or wormwood.

Alternatively and/or additionally, the ingredient Iₙ can be dried, cut or whole parts of a plant approved under food law as a tea and/or tea infusion.

The ingredient is preferably in solid form, more preferably as a powder or granulate. In one embodiment according to the invention, different ingredients can be present as a mixture in various forms.

In another specific embodiment, warnings and/or consumption and/or safety instructions are automatically generated for each ingredient, starting from a threshold value defined for the respective ingredient. These warnings and/or consumption and/or safety instructions can be printed on the packaging during manufacture or issued as a package insert.

In another specific embodiment, minimum and maximum values are defined in advance for each ingredient, so that an individualized composition of a preparation based on the selected attributes is automatically within these minimum and maximum values.

In one embodiment, the parameterization further comprises specific subjective attributes, such as a definition of the label text, one or more colors of label text and/or packaging, one or more colors of a carrier and/or a flavoring or aroma substance in the (gelatin or hydroxypropyl methylcellulose) carrier, or in the preparation.

In a particular embodiment, parameterization comprises a preview function of the preparation, in particular including in the manufacturing any subjective attributes, and/or warnings, and/or consumption and/or safety instructions.

In yet another embodiment, the parameterization comprises a preview function of an individual nutritional table of the ingredients in the preparation, such as quantity, % of the recommended daily value, etc.

In one specific embodiment, the method according to the invention comprises providing a data collection element. This data collection element can, for example, be helpful in measuring and recording data relevant to setting parameters. In a further preferred embodiment the data collection element is designed so as to measure and collect at least one physiological parameter of the consumer. To do this, the data collection element can, for example, be equipped with corresponding sensors and/or an analysis unit. It is particularly preferable to design the data collection element to measure and collect a physiological parameter using a sensor. Suitable sensors for the collection of physiological parameters are, for example, accelerometers, gyroscopes, optical sensors and/or capacitive sensors. Suitable analysis instruments for a data collection element are, for examples, chromatographic and/or fluorescence-based analysis instruments for analytes, for example those from liquids, in particular bodily fluids such as sweat, blood, saliva, urine, stool, semen and/or swabs from mucous membranes.

Such data collection elements are suitable for achieving objective measurements beyond a subjective individual assessment, which are useful for setting parameters and for the compilation of an individual mixture of nutritional supplements. For example, depending on the area of application, a medical/health focus or a sports focus can be chosen. Since the administration of nutritional supplements also has a great preventative effect, for example deficiencies can be identified or an adaptation to a certain life situation and/or a change in life situation made, early on by then individually compiling corresponding supplements based on a measured value.

In a specific embodiment, the method according to the invention further comprises the step of storing measured and/or input data that were used for setting parameters in a database profile. These data can then be evaluated in an additional, particularly preferred step and used to calculate a course of actions as a function of time. This can be particularly useful for identifying an action or effect from a use of supplements. Such time recording is likewise suitable, for example, for planning a specific course of action, such as preparation training for a competition.

In one particularly preferred embodiment, the method according to the invention comprises a step for adapting the personalized manufacturing of a nutritional supplement by adapting the step of defining an ingredient amount to a prior definition of a specific ingredient amount.

In one particular preferred embodiment, the method according to the invention comprises a first defining of an ingredient amount as described above and a second defining of an ingredient amount, which is based on the previously defined ingredient amount. This can be particularly useful when there is a specific deficiency of an ingredient, for example by conducting an initial loading phase after this has been measured or is subjectively expected. A loading phase can, for example, provide for a particularly high dose of a certain ingredient or mixture of ingredients, which then goes into a maintenance phase that maintains a certain titer of the relevant ingredients in the body and reduces the dose.

Such a first and second defining can also be applied if, for example, a dynamic adaptation to certain living conditions is to be made. Thus, for example, when an athlete is preparing for a competition, the amount of ingredients can be adapted to the respective training phase and then dynamically redefined as the training phases change. Likewise, it is conceivable that the ingredients could be adapted in this way based on other living circumstances, such as travel, pregnancy, convalescence, prevention, palliative care, medication use, withdrawal, adjustments to the circadian rhythm, military operations, and the like.

In one particular embodiment, the defining of an ingredient amount of the first ingredient comprises, in addition to first parameters, that are individual for the personalized manufacturing of a preparation, second parameters that are shared with a specific group. These parameters can include, for example, belonging to a specific group of people. Parameters that are assigned, such as to a group of people who pursue a common lifestyle, are particularly suitable. In a particularly preferred embodiment, the defining of an ingredient amount of the first ingredient by means of a number of parameters comprises first parameters, that are individual for the personalized manufacturing of a preparation, and second parameters that are general for a specific group for the manufacturing of a preparation. Another aspect of the present disclosure relates to a system for personalized manufacturing of a nutritional supplement, wherein the nutritional supplement is a preparation of N ingredients. The system comprises an input medium, which is designed to capture a number of parameters, wherein the parameters are individual for the personalized manufacturing of a preparation and wherein each parameter is indicative for one or more attributes of a consumer. The system further comprises a processor, which is designed to calculate ingredient amounts mIₙ based on the parameters for each ingredient Iₙ for a number N ingredients, and wherein the processor is designed to determine a filling amount aIₙ by means of the respective density dIₙ and respective concentration kIₙ of each ingredient. The system further comprises a filling unit for the filling of the mixture of all N ingredients at their respectively determined filling amounts aIₙ into a number of carriers, each with a volume V.

The parameters can be recorded in a context-related and interactive manner. This has the advantage that the parameters can be recorded individually and directly, in order to generate the greatest possible usefulness for the consumer.

The parameters can be recorded in different modules. An expert mode can be provided for more experienced consumers, which provides significantly more information and selection options, but also requires corresponding knowledge for the selection.

The method steps can be carried out or supported by a computer program. With this, commands cause a program to be executed by a computer or a computer system. An input mask for recording parameters can be configured so that the parameters are recorded individually and ultimately lead to personalized manufacturing of the preparation.

A further aspect of the present invention relates to a preparation of a personalized manufacturing of a nutritional supplement. It is particularly preferable that the preparation can be obtained using the method according to invention mentioned in the beginning.

The preparation comprises a closable carrier container. It is particularly preferable that the carrier container has a re-closable cover. The carrier container can, for example, have the shape of a can, and is preferably made of plastic and/or glass.

A re-closable cover can, for example, be connected to the container in a form-fitting manner by means of threading.

The preparation according to the invention further comprises a defined plurality of carriers with a first defined volume. It is particularly preferable that the carriers are fillable capsules that have a volume into which a certain defined amount of ingredients can be filled. The capsules can, for example, be in the form of two-part capsules, which when connected to one another enclose a hollow space that is available as a filling volume.

The preparation according to the invention further comprises a plurality of ingredients with a first density and a first concentration, which together form a total amount of ingredients with a total volume. It is particularly preferred that the plurality of ingredients are present as a homogeneous mixture. The homogeneous mixture, or the plurality of ingredients, is uniformly distributed over the defined plurality of carriers. The defined plurality of carriers is defined depending on the total volume and the defined volume of the carriers.

Under the present invention, the number of carriers can be determined by the total volume of the plurality of ingredients. Thus for example, certain ingredients are available as granules that have a specific grain size. Granules are preferably selected so that they have an ideal surface to volume ratio, which makes rapid absorption of the ingredients possible. Essentially round granulate beads also allow the volume of the carriers to be used optimally.

The preparation according to the invention is further characterized in that the plurality of ingredients are determined by means of a number of parameters, wherein the parameters are individual for the personalized manufacturing of a preparation.

In one specific embodiment, the closable carrier container is designed to be tamper-proof, so that it is possible to recognize that it has been opened a first time. This can be ensured, for example, with a sealing tape or a predetermined breaking point, such that once opened, these will make the opened state of the carrier container clearly recognizable.

In one specific embodiment, the carrier container and/or at least one of the defined pluralities of carriers comprise(s) an recognition means. The recognition means can, for example, have an identification means that makes the preparation and/or individual carrier clearly assignable to a person, i.e. to a consumer who initiated the personalized manufacturing.

One advantage of this assignability is that the personalized preparation is actually only taken by the person for whom it was originally intended and for whom it was individually customized. Suitable identification means can be, for example, QR codes or other optically recognizable elements that are machine-readable. Alternatively and/or additionally, electromagnetic identification means, such as RFID tags, can also be provided.

In one particular embodiment, the preparation according to the invention comprises at least one data collection means, which is suitable for collecting consumer-relevant data and making these available for setting parameters and the resulting compiling of ingredients.

Suitable data collection means are, for example, so-called wearables, i.e. smart watches or sensors, which are worn on the body and measure physiological parameters periodically and/or continuously. Suitable data collection means can, for example, be designed to collect a measured value selected from the group consisting of: blood pressure, blood oxygen saturation, heart rate, breathing rate, pupil size, muscle tone, skin tension, skin moisture and/or skin color to measure. This lets suitable parameters to be defined with the preparation according to the invention, which makes possible an individual composition of ingredients.

Alternatively and/or additionally, the data collection means comprises a means for the ascertaining and evaluating of the body's analytes, i.e. a means for the obtaining, for example, blood, urine, hair, saliva, secretion, stool or other bodily samples and evaluating them with regard to the body's biomarkers. Thus for example, blood tests can be suitable for evaluating the composition of fatty acids in the blood, the supply of elements such as the trace elements zinc, copper, selenium, magnesium, manganese and calcium, the supply of vitamins such as vitamin D3, vitamin E and vitamin A, vitamin B12, vitamin B3, vitamin B5 and/or choline, as well as the supply of essential and non-essential amino acids. Suitable data collection means for the collection of such analytes are preferably minimally invasive and can be used directly by the consumer without the need for medically trained personnel.

Suitable data collection means that operate on the basis of minimally invasive diagnostics are known to a person skilled in the art, and can be selected as needed for the desired implementation of the teaching according to the invention.

Particularly suitable are devices that enable the consumer to draw capillary blood by themselves and essentially painlessly.

In one particular embodiment, the data collection means is designed to establish a communication interface with a database. The communication interface is preferably set up for wireless data transmission and is used to exchange collected measurement values with the database. The database can be designed to log and evaluate these measured values and store them in a traceable manner over time. The solution according to the invention, or the computer program product described above, can be designed to put together a corresponding personalized manufacturing of nutritional supplements based on these data. It would be self-evident to a person skilled in the art that all of the described embodiments can be implemented in a design according to the present invention, provided that they do not explicitly exclude one another.

Below, the present invention will now be explained in more detail using, but not being limited to, specific embodiment examples and figures.

By studying these particular embodiments and figures, further advantageous embodiments of the present invention can be obtained by a person skilled in the art.

### DESCRIPTION OF THE DRAWINGS

Embodiments of the invention are described with reference to the following figures, in which the same reference symbols designate the same or similar parts. These show:

FIG. 1: a schematic sequence of steps according to the method according to the invention;

FIG. 2: a schematic representation according to a method for the personalized manufacturing of nutritional supplements;

FIG. 3: a schematic representation according to the method according to the invention in a further possible embodiment;

FIG. 4: a schematic representation of a computer system comprising a means for carrying out the method according to the invention;

FIG. 5: a schematic implementation of the teaching according to the invention with a series of data collection means;

FIG. 6: a minimally invasive data collection means for the collecting of a blood sample; and

FIG. 7: a schematic representation of a preparation of a personalized manufacturing of a nutritional supplement.

### IMPLEMENTATION OF THE INVENTION

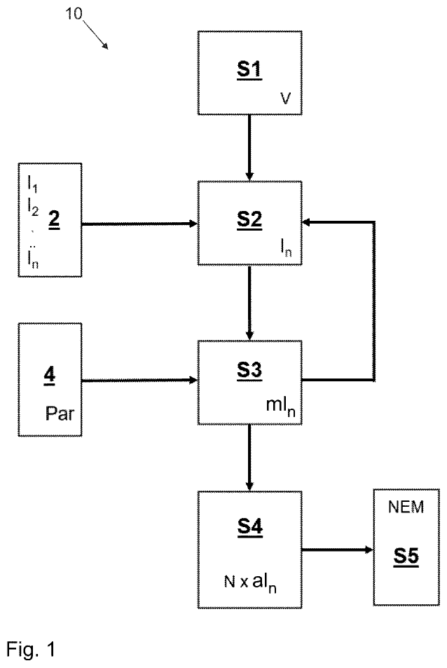

FIG. 1 shows a method 10 with a sequence of steps for personalized manufacturing of a nutritional supplement. In a first step S1 at least one carrier with a first defined volume V is provided. The volume V can be adjusted according to the carrier or carrier type. In another step S2 an ingredient I₁ with a first density dI₁ and a first concentration kI₁ is provided. In the following step S4 an ingredient amount mI₁ of the first ingredient I₁ is defined or determined by means of a number of parameters 4, in which the parameters 4 are individual for the personalized manufacturing of a preparation. Steps S2 and S3 are repeated for a second ingredient I₂ and each additional ingredient Iₙ, according to an ingredient group 2. This is done until a preparation can be made from N ingredients, where N is an integer multiple of 1 and is determined based on consumer input and/or parameters 4. In a further step S4, a manufacturing determination is performed according to the determined total amount of N ingredients. For each ingredient Iₙ, a filling amount aIₙ is determined from the respective ingredient amount mIₙ, divided by the respective density dIn and the respective concentration kIₙ (mIₙ/dIₙ/kIₙ). The sum of all N filling amounts aIₙ is divided by the defined volume V of the carrier (ΣaIₙ/V). Subsequently, in step S5, the ingredients are filled into a respective carrier.

### Example 1

The following shows a compilation of a preparation made up of N ingredients, where N corresponds to a count of 11 ingredients. Two capsules serve as carriers, which together form the amount desired by a consumer.

### Ingredient:

Cholecalciferol 0.071 g
Cyanocobalamin 0.192 g
Calcium d-pantothenate 1.282 g
Menaquinone-7 2.137 g
Black pepper powder 5.344 g
Ginseng root powder 7.5 g
Turmeric root extract 10.687 g
Guarana 10.687 g
L-ascorbic acid 17.1 g
Magnesium oxide 21.267 g
Maltodextrin 21.644 g

This results in a total amount of 95.774 g

The amount is mixed and distributed to 290 capsules. In the present example, each capsule has a volume of 0.68 mL.

This results in a manufacturing of a nutritional supplement with ingredients magnesium oxide, L-ascorbic acid, turmeric root extract, guarana, ginseng root powder, black pepper, menaquinone-7, calcium D-pantothenate, cyanocobalamin, cholecalciferol and the filler maltodextrin. The capsule shell is made of gelatin.

In the present example, a flavoring substance can also be added to give the capsule a flavor, and a capsule color can be defined. In the example given, a batch of red capsules is manufactured with the preparation in question. Another batch for the same consumer is manufactured with green capsules, for example, and yet another in another color. In this way, the consumer can easily distinguish between different nutritional supplements or different expiration dates, for example.

By setting parameters for the selection of ingredients In, the fully automated generation of a list of ingredients is possible, including minimum shelf life and administration recommendations for the desired dose, 2 capsules per day in the example above.

According to the consumption recommendation, an ingredients list would also indicate the following consumption values:
Pantothenic acid 8.55 mg daily ration, therefore 142% of the recommended daily value
Vitamin B12 12.82 mcg daily ration 513% of the recommended daily value
Vitamin C 114.00 mg daily ration 142% of the recommended daily value
Vitamin D3 14.25 mcg daily ration 285% of the recommended daily value
Vitamin K2 28.50 mcg daily ration 38% of the recommended daily value
Magnesium 85.50 mg daily ration 23% of the recommended daily value
Turmeric 71.25 mg daily ration
Black pepper 35.62 mg daily ration
Ginseng 50.00 mg daily ration
Guarana 71.25 mg daily ration

Based on the selection, safety information would be automatically retrieved from a database and, in the following example, this safety information would be generated: “Caffeine is not recommended for children or pregnant women.” “Patients taking anticoagulants should consult their doctor before taking vitamin K supplements.”

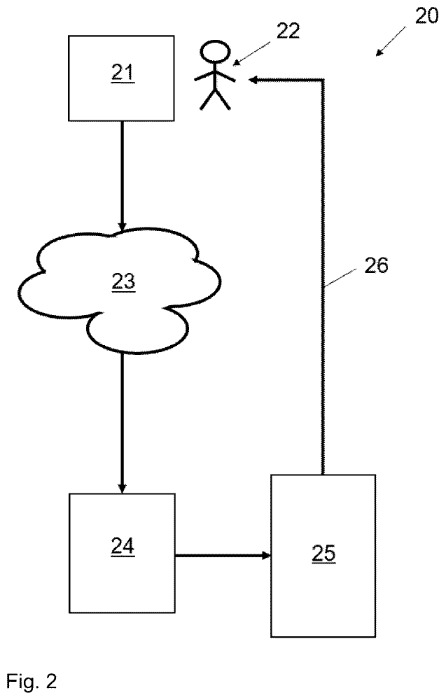

FIG. 2 shows a schematic representation of a method 20 for the personalized manufacturing of nutritional supplements. A consumer 22 enters parameters 4, relating to a nutritional supplement to be customized, using an input and/or query means 21. In query mode, the information or parameters 4 are sent over a network 23, e.g. an intranet or the Internet, to a manufacturing system 24, where the personalized manufacturing of the nutritional supplement takes place according to method 10. The individualized nutritional supplement is crated in a filling device 25 and made available to the consumer 22, who receives it through a channel 26.

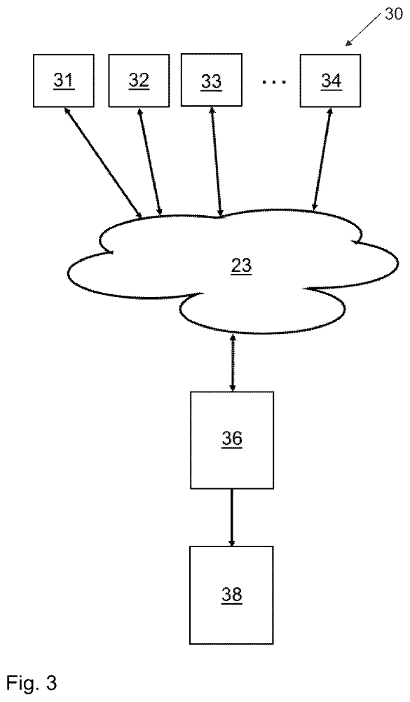

FIG. 3 shows a schematic representation of a system 30 for the personalized manufacturing of nutritional supplements for various consumers or users. A first input and/or query unit 31 for a first consumer, a second input and/or query unit 32 for a second consumer, a third input and/or query unit 33 for a third consumer and additional input and/or query units 34 for additional consumers are connected to network 23. Also connected to network 23 is a manufacturing determination system 36, which determines individualized nutritional supplements for the first, second, third and subsequent consumers. The data for the determined individualized nutritional supplements are passed on to the filling device 38, where the personalized manufacturing of the nutritional supplement takes place for the respective consumer.

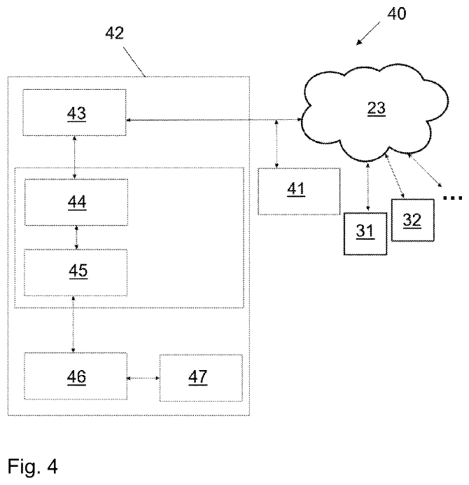

FIG. 4: shows a schematic representation of a computer system 40 comprising a means for carrying out the method according to the invention. Computer system 40 is connected with a network 23, wherein a first input and/or query unit 31, a second input/query unit 32, and/or additional input and/or query units can be connected to the network. Network 23 can correspond in one possible embodiment to a known network type. Computer system 40 comprises a computer or server 42 for manufacturing determination. Computer system 40 further has a database 41, which is connected to server 42 and also to network 23. Server 42 comprises an input and output module 43, at least one processor 44, and a memory 45, which can be volatile and/or non-volatile. Server 42 also has a manufacturing module 46 and a filling module 47. The manufacturing module 46 uses processor 44 to determine relevant parameters 4 for the manufacturing based on consumer information provided by the input and/or query units 31, 32. For each ingredient Iₙ determined, a filling amount aIₙ is specified. This is done by dividing the respective ingredient amount mIₙ by the respective density dIₙ and the respective concentration kIₙ (mIₙ/dIₙ/kIₙ). The sum of all N filling amounts aIₙ is divided by the defined volume V of the carrier (ΣaIₙ/V). Filling module 47 provides all data, including the respective defined ingredients Iₙ. The data can be stored in memory 45 and/or database 41. If the data are stored in database 41, they can be accessed again later by the same consumer. It is also possible to make comparisons or perform comparative analyses for different consumers or users, based on the stored data. This can be done by means of algorithms or artificial intelligence and is used to improve personalized manufacturing.

### Example 2

Example 2 shows an embodiment of an interactive parameter input. Parameters are queried using an input and/or query unit.

To ascertain suitable parameters, a selection of questions can be used through successive dialog boxes. Suitable questions for ascertaining a significant number of parameters according to the invention can be:
sex, age group, weight, weight satisfaction, height, issues to be addressed, joint pain, hay fever, food intolerances, consumption of fish, consumption of fruits and vegetables, sports activity, alcohol consumption, smoking habits, any medications (e.g. blood clotting).

The questions may require the entry of an exact numerical value (e.g. weight in kg), or the selection of a simple yes/no answer or a selectable range (18-35 years), or a subjective state of well being (“I am often tired”).

A system, such as the manufacturing system or manufacturing determination system, determines and creates a nutritional supplement mix based on individual information.

### Example 3

This example shows an embodiment with a personalized result.

Your nutritional supplement mix was
created for you based on your information:
You take 2 capsules a day.
Your nutritional supplement lasts for 145 days.
Your nutritional supplement contains 8 ingredients.
You pay X.- euros per day.

| | | |
|---|---|---|
| Ingredient | Per day | % RDV |

| | | | |
|---|---|---|---|
| Vitamin K2 | 28.29 | mcg | 38 |
| Vitamin B6 | 1.98 | mg | 141 |
| Vitamin D3 | 14.14 | mcg | 283 |
| Spirulina | 14.45 | mg | |
| Turmeric | 70.72 | mg | |
| Sencha green tea | 70.72 | mg | |
| Black pepper | 28.29 | mg | |
| L-carnitine | 424.35 | mg | |

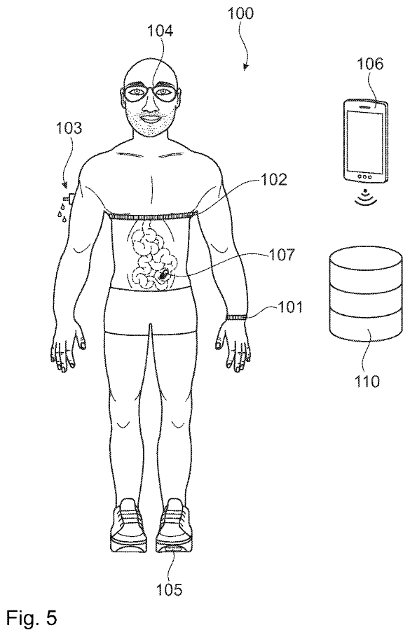

A personalized nutritional supplement mix is provided with information on how to take it, duration of use and ingredients. The ingredients in the preparation indicated in an individualized nutritional table, with quantity and % of the recommended daily values. Likewise, a price can be given for each ingredient and for the mix of the individual ingredients (the latter not shown). FIG. 5 shows an example of a consumer 100, who uses a plurality of different data collection means to collect a series of physiological measurement data, which can be used to determine parameters according to the invention.

It goes without saying that for a person skilled in the art, the selection of the data collection means shown is an example, and could be combined as desired in an implementation of the teaching according to the invention.

For example, consumer 100 uses a fitness tracker 101 as a wearable. This fitness tracker 101 is, for example, suitable for detecting an recording movements, so that calorie consumption can be determined. Modern fitness trackers also have the means to at least approximately determine blood oxygen saturation and heart rate. The fitness tracker can be a separate element or part of a smartwatch with a fitness tracker function. It is particularly preferred for the fitness tracker 101 to be integrated into a smartwatch, and the teaching according to the invention with the computer program product indicated above can, for example, have an app implementation that is directly connected to the parameter setting of the personalized ingredients. Recorded data can therefore be recorded more or less in real time and used to put together a personalized manufacturing of ingredients. This is particularly suitable for athletes, such as in preparation of a competition. This means that supplements can be manufactured in relatively small batches, which adapt dynamically to the training process and these adaptations reconfigured and made available to the consumer. Integration with a smartphone 106 is particularly suitable. Smartphones 106 are ubiquitous in modern society and therefore suitable data collection means for the collection of personal data. An app connection under the present invention can, for example, also provide for the questions mentioned above used for setting parameters being repeated at periodic intervals, or that additional questions are asked by the app at different intervals, as messages or prompts to the consumer, in order to subsequently carry out, for example, a fine adjustment of the parameter settings and/or a dynamic adaptation of a second manufacturing configuration. This can also, for example, be done through feedback, so that after a first preparation, a second preparation is put together, depending on the feedback from the first preparation.

It is particularly preferable that data are recorded for this purpose and stored in a database 110. Database 110 can be hosted on a server and access can only be allowed for authorized users by means of appropriate security measures. The database can also be operationally connected to a machine learning system, which makes individual adjustments to the parameter settings based on globally recorded metadata, which can be formed, for example, from the totality of consumers using the teaching according to the invention.

In another possible embodiment, the consumer 100 also uses smartglasses 104 as a wearable. Smartglasses 104 can, for example, be designed to record pupil size, eye moisture, sunlight, pupil movements or other, sometimes autonomically controlled health parameters, and to make these data available, like with the data from the smartwatch described above, to database 110 for evaluation.

These smartglasses 104 can also be designed with suitable sensors, for example for detecting head and body movements, using accelerometers and/or gyroscopes.

The consumer shown also has, as an example, a heart rate belt 102. Heart rate belts are used by athletes to accurately measure their heart rate during training and/or to train specifically at a certain heart rate range, for example. Modern heart rate belts can be connected to smartphones so that the data can be accordingly evaluated. Conclusions can be drawn from these heart rate data about, such as calorie consumption, that are more precise than simply calculating the number of kilometers covered. This calorie consumption can, for example, be used as an indication for supplementing with certain ingredients for setting parameters according to the present invention.

The consumer 100 shown also has a pedometer 105 in his shoe. Pedometer 105 is suitable for detecting and measuring impacts or steps by means of accelerometers or gyroscopes, respectively, and for transmitting these wirelessly to an evaluation unit, a smartphone 106 in this example.

In addition to these externally measured fitness data, a blood sample from consumer 100 can be used to determine, for example, a certain nutrient level in the body, and to provide this for the setting of parameters for the ingredients. It has been shown that a blood draw that is as minimally invasive as possible is beneficial for better and broader acceptance. In the present example consumer 100 therefore uses a minimally invasive blood draw means 103. Minimally invasive blood draw means 103 can be designed to take just a sufficient amount of capillary blood from consumer 100, which is suitable for determining the desired blood values, which are used as parameters for defining ingredient amounts.

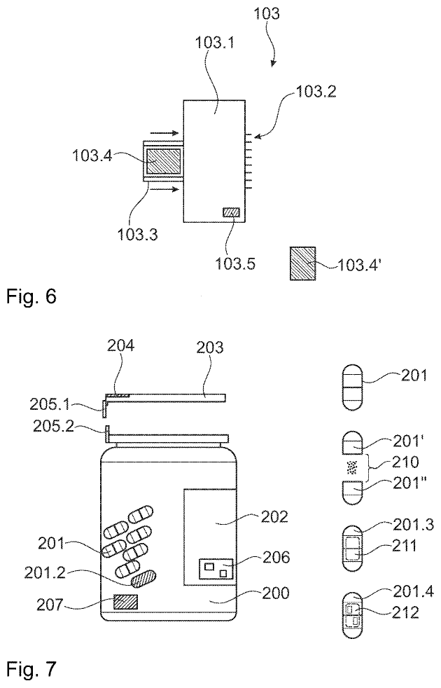

A suitable device for the minimally invasive blood draw 103 is shown in FIG. 6 as an example. The minimally invasive blood draw means 103 comprises a body module that serves as a carrier for the functional units and as a support for the blood collection site. The upper arm has proven to be particularly suitable as a blood draw site, as it is broadly accessible and comparatively less sensitive to pain. The thigh, buttocks and calf are also suitable. However, for the application shown, the upper arm is intended, which is perforated by an arrangement of microneedles 103.2, and by creating a vacuum, blood is drawn out of the capillaries. In order to collect the blood, a collection container 103.4 is guided by means of a slide 103.3 through a sealing nipple (not shown) in operational connection to the vacuum so that the blood is drawn. A small and just sufficient amount of blood is preferably collected in this way.

The filled collection container 103.4′ can, for example, be brought or sent to a suitable laboratory for evaluation purposes. The collection container can contain suitable reagents and/or other buffers for stabilizing and/or fixing the blood sample.

FIG. 7 shows a preparation according to the invention, as can be obtained from carrying out a method according to the invention.

The preparation comprises a carrier container 200, which is designed to accommodate a plurality of carriers 201. In the present example, carriers 201 are designed as capsules, which consist of a first carrier half 201′ and a second carrier half 201″. The capsules 201 in the example are made of gelatin and the two carrier halves 201′ and 201″ are pushed into one another and locked together so that they form an internal volume that is designed to accommodate an ingredient mixture 210. In the present example, ingredient mixture 210 is designed as a powder and/or granulate, but an ingredient mixture 210 that consists at least partially of a liquid and/or a dispersion is also conceivable, e.g. a gel and/or a solid, and oil and/or an aqueous solution, an alcohol, an ester or a combination thereof, depending on the specific composition of the ingredients and suitable carrier medium for the ingredients, which are filled into the corresponding carrier 201.

In the present example from FIG. 7, alternative carriers 201.2, 201.3 and 201.4 are also shown. A special carrier 201.2 can, for example, be identified by a special color in carrier container 200. Special carrier 201.2 can have a different ingredient composition 210 or can only be differentiated form the others by color. In an embodiment according to the invention, a plurality of such special carriers 201.2 can be provided in carrier container 200. It is advantageous, for example, if fats are taken in addition to water for certain vitamins, since these vitamins are essentially fat-soluble. Special carriers 201.2 may then be made available to provide, for example, a supply of fat in the form of, for example, gelatin-coated oil capsules or similar administration means known to a person skilled in the art.

In the present example, a probe carrier 201.3 is also provided. Probe carrier 201.3 encloses a probe within itself, which is suitable for collecting measured values from the user's intestines. Referring again to FIG. 5, consumer 100 also uses a probe 107, which measures, for example, certain intestinal parameters, such as pH value, or simply the motility of the intestines. The motility of the intestines can easily be measured using, for example, a probe that essentially passes through the intestine and is used to record the time between ingestion of the probe and the excretion of it. It is particularly preferable for the probe to be designed for this purpose in such a way that it essentially passes through the intestine easily and is easily recognizable in the stool.

Capsule endoscopy analyses of the intestines are also conceivable, for example, to detect inflammation or other changes in the intestines and to determine corresponding parameters for the manufacturing of ingredients according to the present invention.

In the present example, identification holders 201.4 are also provided. This identification holders 201.4 can have optically recognizable elements on their surface, for example in the form of a printed capsule label 212. The capsule label can, for example, be provided with marks that can lead to information being reproduced by an optical detection means. Suitable marks can be recognized by optical cameras, such as on smartphones, in a way similar to QR codes, and lead to the reproduction of information through access to a database.

It is also possible that all carriers 201 in a carrier container according to the invention are provided with such identifications 212. These capsules can be clearly and unambiguously assigned to the personalized manufactured product according to the present invention even after they have been removed from carrier container 200, so that the consumer can always be sure that he or she is using specific capsules and can rely on the capsules containing the ingredient mixtures he or she has put together with the aid of the parameter settings.

In the present example, container 200 is closed with a container cover 203. Container cover 203 also has an identification means 204. Identification means 204 can, for example, be a machine-readable identification means 204. Particularly suitable are passive tags, such as passive RFID tags, which can be activated and ready using an RFID reader. On the one hand, this can be used for the logistics of the corresponding packaging, i.e. the correct delivery of the personalized manufactured product to the correct consumer 100, but alternatively also for the consumer to identify the mixture specially put together for him or her.

In the present example, container cover 203 also has a first part 205.1 for tamper protection. A second part 205.2 for tamper protection is attached to the edge of container 200, which is to be brought into operational connection to cover 203. In a simple embodiment, these tamper protections 205.1 and 205.2 are designed as a seal, which tears when the container is opened for the first time and cover 203 is removed, thus making tampering with the container obvious.

In the present example, container 200 according to the invention also has an aroma element 207 introduced into the container or simply inserted therein. Aroma element 207 is used to flavor the capsules with a specific flavor. It has surprisingly been shown that the capsules can be individually flavored and that it is sufficient to insert an aroma element 207 that is, for example, soaked with a specific aroma substance, into container 200 and the capsules are able to absorb this flavor through the gelatin. A suitable flavoring can, for example, impart the flavor of peppermint, citrus fruit, lavender, etc. to the capsules, depending on the consumer's preferences and corresponding parameter setting.

For unambiguous and clear identification of the container 200 and its contents, container 200 also has container label 202, which in turn is equipped with a label marking 206. This label marking 206 can again have a machine-readable optical element, such as a QR code or optical elements that are transferred from an app to a specific virtual environment by means of linking or augmented reality. The present disclosure shows a method and system for the personalized manufacturing of nutritional supplements. It goes without saying that numerous other embodiments can be conceived by a person skilled in the art on the basis of the example embodiments described.

### LIST OF REFERENCE SYMBOLS

S1 First step, preparing carrier
S2 Second step, preparing ingredient
S3 Third step, defining, determining
S4 Fourth step, manufacturing
S5 Fifth step, filling
2 Ingredient
4 Parameters
21 Input and/or query medium
22 Consumer
23 Network
24 Manufacturing system
25 Filling device
26 Channel
30 System
31-34 Input and/or query unit
36 Manufacturing determination system
38 Filling device
40 Computer system
42 Computer or server
41 Database
43 Input and output module
44 Processor
45 Memory
46 Manufacturing module
47 Filling module
100 Consumer with various data collection means
101 Wearable, e.g. fitness tracker
102 Wearable, e.g. heart rate belt
103 Minimally invasive blood collection means
103.1 Body module
103.2 Needles
103.3 Slider
103.4 Collection container
103.4′ Filled collection container
103.5 Trigger
104 Wearable, e.g. smart glasses
105 Wearable, e.g. pedometer
106 Smart phone
107 Probe
200 Carrier holder
201 Carrier
201′ First carrier half
201″ Second carrier half
201.2 Special carrier
201.3 Probe carrier
201.4 Identification holder
202 Label
203 Cover
204 Identification means
205.1 Tamper protection/Seal for first part
205.2 Tamper protection/Seal for second part
206 Label markings
207 Aroma element
210 Ingredient mixture
211 Probe
212 Capsule label
N Number of ingredients
V Volume
aIₙ Filling amount
mIₙ Ingredient amount
dIₙ Density
kIₙ Concentration

1. A method for the personalized manufacturing of a nutritional supplement, in which the nutritional supplement is a preparation of N ingredients, and Nis an integer multiple of 1, comprising the following steps:
a) preparing at least one carrier with a first defined volume V;
b) preparing a first ingredient I₁ with a first density dI₁ and a first concentration kI₁;
c) defining an ingredient amount mI₁ of the first ingredient I₁ by means of a number of parameters, in which the parameters are individual for the personalized manufacturing of a preparation;
d) repeating steps b) and c) for a second ingredient I₂ and each additional ingredient Iₙ, and in which the manufacturing comprises a filling of a total amount of ingredients, where for each ingredient Iₙ a filling amount aIₙ is determined from the respective ingredient amount mIₙ, divided by the respective density dIₙ and the respective concentration kIₙ (mIₙ/dIₙ/kIₙ) and total of all N filling amounts aIₙ is divided by the defined volume V of the carrier (ΣaIₙ/V).

2. The method according to claim 1, wherein the defining of the ingredient amount mIₙ is defined using parameters, which are determined depending on consumer input.

3. The method according to claim 1, wherein the parameters are indicative or one or more attributes or a consumer for whom the preparation is made.

4. The method according to claim 1, wherein each parameter is indicative for one or more attribute(s) selected from the group comprising: age, sex, body mass index, dietary habits, sleep rhythm, working hours, media consumption, leisure behavior, weight, height, skin color, eye color, allergies and/or intolerances, resting heart rate, blood oxygen saturation, cortisol levels, drug and/or stimulant use, blood count information, blood sugar, movement and/or activity data (e.g. calorie consumption) from wearables.

5. The method according to claim 1, wherein N is a whole number between 2 and 20, preferably between 7 and 15, and more preferably is 11.

6. The method according to claim 1, wherein the filling of the total amount of N ingredients takes into account a flavor admixture.

7. The method according to claim 1, wherein one or more ingredients Iₙ are selected so that they correspond to a percentage dose of a recommended daily consumption amount.

8. The method according to claim 1, wherein the ingredients Iₙ are selected from the group comprising: cholecalciferol, cyanocobalamin, calcium D-pantothenate, menaquinone-7, black pepper powder, ginseng root powder, turmeric root extract, guarana, L-ascorbic acid, magnesium oxide, maltodextrin, biotin, folate, niacin, pantothenic acid, riboflavin (vitamin B2), thiamine (vitamin B1), vitamin B6, vitamin B12, vitamin C, vitamin D3, vitamin E, calcium, magnesium, turmeric, ginger, fenugreek, green tea, beta carotene (provitamin A), vitamin K2, iron, selenium, zinc, copper, iodine, chromium, molybdenum, manganese, potassium, fluoride, chloride, phosphorus, lactase, L-arginine, L-glutamine, L-histidine, L-lysine, coenzyme Q10, caffeine, glucosamine, creatine, L-carnitine, taurine, garlic, black pepper, maca, spirulina, cannabidiol, ginseng, oligomeric proanthocyanidins, guarana, echinacea, green coffee, barley grass, saffron and L-tryptophan, in particular in forms administered as a dietary supplement.

9. The method according to claim 1, wherein a probiotic is used as ingredient Iₙ.

10. The method according to claim 1, wherein the carrier is a capsule that preferably comprises gelatin.

11. The method according to claim 1, wherein the carrier is provided as a tablet, pill, gummy, bottled drink and/or effervescent tablet.

12. A system for the personalized manufacturing of a nutritional supplement, wherein the nutritional supplement is a preparation made from N ingredients, comprising:
an input medium, which is designed to capture a number of parameters, wherein the parameters are individual for the personalized manufacturing of a preparation and wherein each parameter is indicative for one or more attributes of a consumer;
a processor, which is designed to calculate ingredient amounts mIₙ based on the parameters for each ingredient In for a number N ingredients, and wherein the processor is designed to determine a filling amount aIₙ by means of the respective density dIₙ and respective concentration kIₙ of each ingredient; and
a filling unit for the filling of the mixture of all N ingredients at their respectively determined filling amounts aIₙ into a number of carriers, each with a volume V.

13. The system according to claim 12, wherein the capture of the parameters is context dependent and takes place interactively.

14. A computer program, comprising instructions that, when the program is executed by a computer, causes the computer to carry out steps from the method according to claim 1.

15. The computer program according to claim 14, wherein an input mask is configured and the parameters define the personalized manufacturing of the preparation.

16. A preparation from a personalized manufacturing of a nutritional supplement, in particular obtainable by a method according to claim 1, comprising:
a. a closable carrier container;
b. a defined plurality of carriers with a first defined volume V;
c. a plurality of ingredients I₁, with a first density dI₁ and a first concentration kI₁, which together form a total amount of ingredients with a total volume, and in particular as homogeneous mixtures, are uniformly distributed over the defined plurality of carriers, wherein the defined plurality of carriers is defined depending on the total volumes and the defined volume V;
characterized in that the plurality of ingredients are determined by means of a number of parameters, wherein the parameters are individual for the personalized manufacturing of a preparation.
